(19) United States
(12) Patent Application Publication
(10) Pub. No.: US 2012/0196358 A1
(43) Pub. Date: Aug. 2, 2012

(54) DEVICE FOR REMOVING CUMULUS FROM OOCYTES

(76) Inventors: Fred Burbank, Laguna Niguel, CA (US); Michael Jones, San Clemente, CA (US)

(21) Appl. No.: 13/322,886

(22) PCT Filed: May 27, 2010
(86) PCT No.: PCT/US10/36401

§ 371 Date: Apr. 17, 2012

Related U.S. Application Data
(60) Provisional application No. 61/182,981, filed on Jun. 1, 2009.

(51) Int. Cl. C12N 5/075; C12M 1/12

(52) U.S. Cl. 435/325; 435/283.1

(57) ABSTRACT

Disclosed herein are devices, methods, and kit of parts adapted for stripping cumulus from a plurality of oocytes contained therein.

### FIELD OF THE INVENTION

The invention relates to devices adapted for stripping cumulus from oocytes contained therein and methods for using the device.

### BACKGROUND

In vitro fertilization (IVF) and embryo transfer are a commonly practiced treatment for a variety of causes of infertility. IVF is a laboratory process where infertile women are treated by a physician with the intent to become pregnant. The practice is highly specific and involves hormone therapy to stimulate the ovaries and subsequent harvest of the oocytes by the physician via intra-vaginal ultrasound. Agricultural industries may also rely upon such assisted reproduction techniques. In bovine, embryo transfer may result in a higher pregnancy rate. However, low fertilization rate in some patients and a low implantation rate per embryo can be cause of frustration and emotional distress. Expense and relatively low success rates can place significant burden on the use of these assisted reproduction techniques for humans as well as livestock. In human reproduction, such expense and failure can add emotional as well as economic burdens.

Failure rate in reproduction techniques may be attributable to handling and preparation of oocytes or eggs for manipulation while executing reproduction techniques. The oocytes that are harvested come surrounded by a mass of cells called the cumulus or cumulus mass. IVF that takes place by means of intracytoplasmic sperm injection (ICSI), involves the removal of the cumulus and corona cells surrounding the oocyte. Typically, an enzyme hyaluronidase from bovine origin is used for oocyte denudation (removal or stripping of cumulus). This enzyme may digest the hyaluronic acid that is interspaced between the cumulus cells, thus liberating the oocyte for maturity grading and microinjection. The methods are typically conducted by mechanical denudation or decoronization by means of multiple pipetting.

Typical methods utilize a sharpened pipette to dissect the cumulus mass off the egg or a combination of a hyaluronidase enzyme to dissolve the mass and pipette transfer to shear to soften mass off the egg. For example, Nagy, Z. P. et al. Fertility and Sterility 85(5):1544-1546 (2006), describe the cumulus stripping process where a low concentration of enzyme is used to limit the influence of any toxins that may be present in the enzyme. In order to improve the removal of cumulus, the cumulus-oocyte complex (COCs) are pipetted up and down until the partial removal of the COCs takes place. See page 1544, right hand column, last paragraph. After the partial removal of the COCs, the oocytes are rinsed and are subjected to mechanical pipetting again for a complete removal of COCs from the oocytes. See page 1545, left hand column. De Vos, A. et al. Human Reproduction 23(8):1815-1819 (2008), describe pipetting of the oocytes in and out of the pipette for about 16-17 times for mechanical denudation or decoronization. See page 1817, right hand column, first paragraph and Table I.

The methods requiring mechanical pipetting may require a relatively large amount of the enzyme followed by back and forth transfer between pipettes to shear the mass off the egg. The relatively high concentrations of the enzyme may lead to contamination of the oocytes due to higher impurity and a higher pathogen concentration in the enzymes if the enzymes are animal-derived products. Further, the time required for total denudation may be long since multiple pipetting may be required for total denudation. Furthermore, the use of pipetting or multiple pipetting may lead to damage or trauma to the oocyte resulting in non-viable oocytes or failed IVFs.

Therefore, there is a need for an oocyte handling device and method adapted for stripping cumulus from oocytes contained therein.

### SUMMARY OF THE INVENTION

In one aspect, there is provided a device adapted for stripping cumulus from a plurality of oocytes contained therein, which device comprises:
a tube; and
a filter affixed inside the tube, which filter comprises a plurality of pores having a diameter smaller than a diameter of said oocytes but larger than an individual cells of cumulus thereby restricting passage of said oocytes through the filter while permitting passage of the cumulus cells there through.

In one aspect, there is provided a device adapted for stripping cumulus from a plurality of oocytes contained therein, which device comprises:
a tube having a constriction; and
a filter affixed inside the tube at the point of the constriction, which filter comprises a plurality of pores having a diameter smaller than a diameter of said oocytes but larger than an individual cells of cumulus thereby restricting passage of said oocytes through the filter while permitting passage of the cumulus cells there through.

In one aspect, there is provided a device adapted for stripping cumulus from a plurality of oocytes contained therein, which device comprises:
a tube having a wider center portion; and
a filter affixed inside the wider center portion of the tube, which filter comprises a plurality of pores having a diameter smaller than a diameter of said oocytes but larger than an individual cells of cumulus thereby restricting passage of said oocytes through the filter while permitting passage of the cumulus cells there through.

In another aspect, this invention provides a method for stripping cumulus from a plurality of oocytes which method comprises:
1) placing the plurality of oocytes on a filter affixed inside a tube, wherein the filter comprises a plurality of pores wherein the pores have a diameter smaller than a diameter of the oocytes but larger than an individual cells of cumulus thereby restricting passage of said oocytes through the filter while permitting passage of the cumulus cells there through; and
2) flowing an effective amount of solution comprising a cumulus stripping agent over the oocytes for a time sufficient to strip the cumulus from the oocytes.

These and the other embodiments are further described in the text that follows.

### BRIEF DESCRIPTION OF THE DRAWINGS

This invention will be further described with reference being made to the accompanying drawings.

FIG. 1A illustrates a device embodiment of the invention.

FIG. 1B illustrates a cross-sectional view of the device embodiment of the invention.

FIG. 2 illustrates other examples of the device embodiment of the invention..

FIG. 3 illustrates a device containing multiple sieves and a filter.

FIG. 4 is a flow chart illustrating a method embodiment of the invention.

FIG. 5 shows the five oocytes with the cumulus before the oocytes are subjected to the cumulus stripping method using the device (Example 1).

FIG. 6 shows the five oocytes after stripping of the cumulus (Example 1).

FIG. 7 shows the ten oocytes with the cumulus before the oocytes are subjected to the cumulus stripping method using the device (Example 2).

FIG. 8 shows the ten oocytes after stripping of the cumulus (Example 2).

FIG. 9 shows the fifteen oocytes with the cumulus before the oocytes are subjected to the cumulus stripping method using the device (Example 3).

FIG. 10 shows the fifteen oocytes after stripping of the cumulus (Example 3).

### DETAILED DESCRIPTION OF THE INVENTION

Before the devices, methods, and kit of parts are described, it is to be understood that the invention is not limited to the particular methodologies, protocols, and reagents described, as these may vary. It is also to be understood that the terminology used herein is intended to describe particular embodiments of the present invention, and is in no way intended to limit the scope of the present invention as set forth in the appended claims.

### 1. Definitions

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

In accordance with the present invention and as used herein, the following terms are defined with the following meanings, unless explicitly stated otherwise.

The term “about” when used before a numerical designation, e.g., pH, temperature, amount, concentration, and molecular weight, including range, indicates approximations which may vary by (+) or (−) 5%, 1% or 0.1%.

As used in the specification and claims, the singular form “a”, “an” and “the” include plural references unless the context clearly dictates otherwise. For example, the term “a cumulus cell” includes a plurality of cumulus cells or the term “oocyte” includes a plurality of oocytes.

As used herein, the term “comprising” or “comprises” is intended to mean that the devices and methods include the recited elements, but not excluding others. “Consisting essentially of when used to define devices, methods, or kit of parts, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace amount of elements of other ingredients and substantial method steps. Embodiments defined by each of these transition terms are within the scope of this invention.

The term “cumulus stripping agent” as used herein refers to any agent that partially or completely strips or removes cumulus cells from the oocyte.

The term “cumulus” or “cumulus cell” as used herein refers to cells that surround the oocyte.

The term “oocyte” as used herein is meant to be synonymous with the term “egg.” The oocyte includes mature animal oocytes and stabilized oocytes.

The term “mature animal oocytes” refers to harvested oocytes which are graded on a maturation scale as “mature stage—MII.” This scale further identifies harvested oocytes as “intermediate stage—(MI)” or “immature stage—(GV)”.

The term “stabilized oocytes” refers to mature oocytes still retaining the cumulus mass (granulosis cells) which permit maturation of the oocyte by nutrient intake through gap junctions in the cumulas mass. The mature oocyte is characterized by formation of the meiotic spindle in conjunction with extrusion of the first polar body while maintaining the integrity/activity of the intracellular proteins.

The term “stripping” used herein is synonymnous with the terms “denudation” or “decoronization” which refers to removal of cumulus cells from the oocyte.

### 2. Device

Disclosed herein is a device adapted for stripping cumulus from a plurality of oocytes contained therein. The device comprises a filter affixed inside the device. In general, the device holds a plurality of oocyte which are continuously or intermittently washed with a cumulus stripping agent that strips the cumulus cells off of the oocytes. The filter affixed inside the device comprises a plurality of pores wherein the pores have a diameter smaller than a diameter of the oocytes but larger than individual cells of the cumulus thereby restricting passage of the oocytes through the filter while permitting passage of the cumulus cells there through.

The device disclosed herein prevents manual handling of the oocytes, such as, mechanical pipetting, during the stripping of the cumulus thereby providing a safe, rapid and efficient method for stripping cumulus off the oocytes for their use in reproduction techniques. The device prevents or minimizes a damage that may be caused to the oocytes by the manual stripping of the cumulus cells thereby resulting in more viable oocytes or a higher number of more viable oocytes for fertilization. This, in turn, is contemplated to result in higher number of successful implantations and pregnancies. Owing to minimum manual handling during the cumulus stripping process, the total time taken by the devices of the invention for stripping of the cumulus is less as compared to the process which requires manual handling. It is also contemplated that the devices disclosed herein will reduce the amount of the cumulus stripping agent required for partial or complete removal of the cumulus from the oocytes thereby reducing the contamination of the oocyte and increasing their viability. Another advantage of the devices of the invention is that the processing of the oocytes can be handled as a group as opposed to individually. All oocytes harvested from a patient can be placed into the device containing a solution of hyaluronidase enzyme and then processed as a group. The processing may take about 1-2 minutes for the enzyme degradation and then an additional about 1-2 minutes to complete the entire batch of the oocytes versus about 2 to 3 minutes per oocyte with the manual method.

Accordingly, in one aspect, there is provided a device adapted for stripping cumulus from a plurality of oocytes contained therein, which device comprises:
a tube; and
a filter affixed inside the tube, which filter comprises a plurality of pores having a diameter smaller than a diameter of said oocytes but larger than an individual cells of cumulus thereby restricting passage of said oocytes through the filter while permitting passage of the cumulus cells there through.

In another aspect, there is provided a device adapted for stripping cumulus from a plurality of oocytes, which device comprises:
a tube having a constriction; and
a filter affixed inside the tube at the point of the constriction, which filter comprises a plurality of pores having a diameter smaller than a diameter of said oocytes but larger than an individual cells of cumulus thereby restricting passage of said oocytes through the filter while permitting passage of the cumulus cells there through.

In yet another aspect, there is provided a device adapted for stripping cumulus from a plurality of oocytes contained therein, which device comprises:
a tube having a wider center portion; and
a filter affixed inside the wider center portion of the tube, which filter comprises a plurality of pores having a diameter smaller than a diameter of said oocytes but larger than an individual cells of cumulus thereby restricting passage of said oocytes through the filter while permitting passage of the cumulus cells there through.

In some embodiments, there is provided a device adapted for stripping cumulus from a plurality of oocytes, which device comprises a filter affixed inside the tube, which filter comprises a plurality of pores having a diameter smaller than a diameter of said oocytes but larger than an individual cells of cumulus thereby restricting passage of said oocytes through the filter while permitting passage of the cumulus cells there through.

In some embodiments, the tube is hollow. In some embodiments, the tube comprises an open proximal end, an open distal end and a center portion with a lumen running from the proximal to the distal end of a defined diameter which lumen permits flow of a fluid through said device wherein the diameter of the center portion is narrower as compared to the diameter of the distal end providing the constriction. In another embodiment, the open distal end of the tube can be affixed with a stopper or other device which temporarily halts the flow through the tube. Such a stopper would allow for incubation of the oocytes for a set period of time so as to enhance cumulus removal.

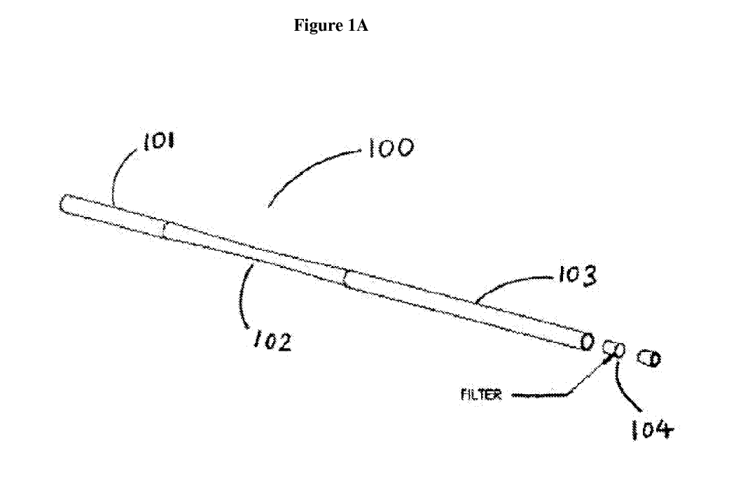

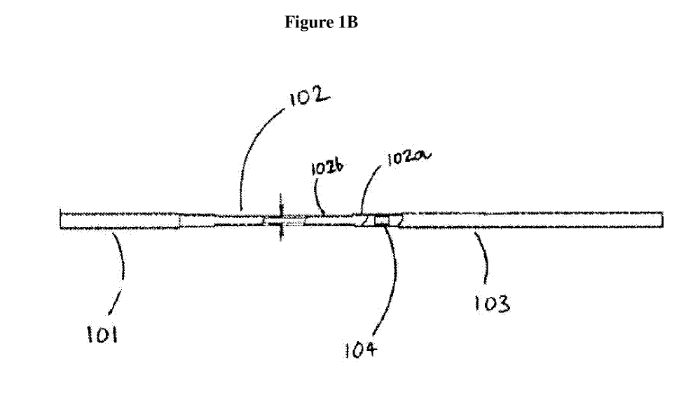

One embodiment of the device is as shown in FIG. 1A. FIG. 1B illustrates a cross-sectional view of the device. The device comprises a tube 100 which comprises an open proximal end 101, a center portion 102, and an open distal end 103. The tube 100 comprises a lumen (not visible in FIG. 1A) that runs from the proximal end 101 through the center portion 102, to the distal end 103 of the tube. The tube further comprises a filter 104 affixed inside the tube 100. In some embodiments, the filter affixed in the tube is replaceable, i.e., the filter may be taken out of the tube and be replaced with a new filter. The filter comprises a plurality of pores that have a diameter smaller than a diameter of the oocytes but larger than an individual cell of the cumulus. This restricts passage of the oocytes through the filter while permitting passage of the cumulus cells there through.

In some embodiments, the center portion holds the plurality of the oocytes. In some embodiments, the proximal end of the device holds the plurality of the oocytes. In some embodiments, the filter holds the plurality of the oocytes. In some embodiments, the plurality of oocytes are mammalian oocytes. In some embodiments, the mammalian oocytes are human oocytes. Mammals include, but are not limited to, murines, rats, simians, humans, farm animals, sport animals and pets.

In an alternative embodiment of the device, as shown in FIG. 1A, a center portion of the tube is wider than the proximal or distal end of the tube and the filter is affixed inside the wider center portion of the tube.

The total length of the tube may be from about 2 inches (5.1 cm) to about 10 inches (25.4 cm) long. In some embodiments, the length of the tube is from about 2 inches to 8 inches; from about 2 inches to 6 inches; from about 2 inches to 5 inches; from about 2.5 inches to 5.5 inches; from about 3 inches to 4.5 inches; from about 3 inches to 4 inches; or from about 3 inches to 3.5 inches. In some embodiments, the length of the tube is about 3.3 inches. The length of the proximal end to the filter (or center portion) may be from about 0.5 inches to about 1 inch. In some embodiments, the length of the proximal end to the filter may be from about 0.5 inches to about 0.7 inches; from about 0.55 inches to about 0.65 inches; from about 0.6 inches to about 0.65 inches; from about 0.6 inches to about 0.7 inches; or from about 0.65 inches to about 0.7 inches. In some embodiments, the length of the distal end from the filter is from about 1 inch to about 3 inches; from about 1 inch to about 2 inches; or from about 1.5 inches to about 2 inches. In some embodiments, the length of the distal end is about 1.5 inches. In some embodiments, the length of the center portion is from about 0.5 inches to about 1.5 inches; or from about 1 inches to about 1.5 inches. In one embodiment, the length of the proximal end 101 is about 0.65 to 0.7 inches; length of the distal end 103 is about 1.5 inches; and the length of the center portion 102 is about 1 inch.

It is to be understood that the optimization of the length of the tube, the length of the proximal end, the length of the open end, or the length of the center portion may depend on the amount of the solution used for the stripping of the cumulus, the amount of oocytes, or the desired length of tube etc. Such optimization is well within the skill of a person of ordinary skill in the art.

The proximal end and the distal end may be of a defined diameter in such a way that a diameter of the center portion is narrower than the diameter of the proximal end of the tube. In some embodiments, the proximal end and the distal end are of a defined diameter in such a way that a diameter of the center portion is narrower than the diameter of the distal end of the tube. In another embodiment, the proximal end and the distal end are of a defined diameter in such a way that a diameter of the center portion is narrower than the diameter of the proximal end of the tube as well as the diameter of the distal end of the tube. In an alternative embodiment, the diameter of the center portion is wider than the diameter of the distal or the proximal end of the device.

In some embodiments of the device as shown in FIGS. 1A and 1B, the diameter of the center portion 102 is narrower than the diameter of the proximal end 101 of the tube as well as the diameter of the distal end 103 of the tube. In some embodiments, the inner diameter of the proximal end as well as the distal end is from about 0.03 to about 0.07 inches, from about 0.04 to about 0.05 inches or from about 0.04 to about 0.045 inches. In some embodiments, the inner diameter of the center portion is from about 0.01 to about 0.05 inches; from about 0.02 to about 0.04 inches; or from about 0.03 to about 0.04 inches. In one embodiment, the inner diameter of the proximal end 101 as well as the distal end 103 is about 0.047 inches and the inner diameter of the center portion 102 is about 0.038 inches. Without limited by any theory, the diameter of the proximal end and the distal end may be different from each other. For example, the diameter of the proximal end may be greater than the diameter of the distal end or vice versa. In the former case, the flow through the filter will be reduced by the narrower distal end thereby creating a longer residence time of the solution in contact with the oocytes.

In some embodiments, the filter 104 in the tube 100 is affixed in the distal end 103 of the tube at the junction between the center portion 102 and the distal end 103 (FIG. 1B). The filter 104 is affixed in its position by virtue of the diameter gradient between the center portion 102 and the distal end 103 where the narrower center portion 102 prevents sliding of the filter 104 inside the tube from the distal end 103 through the center portion 102 to the proximal end 101 or from the center portion 102 to the distal end 103 and out of the tube. It is to be understood that any means that prevent sliding of the filter inside the tube may be employed in the device of the present invention. In such a situation, the diameter gradient between the center portion and the proximal and distal ends may not be warranted. For example, the filter may be affixed in the tube with a tube extrusion that may tighten the fixation of the filter in the tube thereby preventing its sliding.

In some embodiments, the center portion 102 may have two diameter gradients, as shown in FIG. 1B. The center portion 102 comprises a first portion 102a and a second portion 102b where the inner diameter of the first portion 102a is greater than the inner diameter of the second portion 102b. In some embodiments, the inner diameter of the second portion 102b is from about 0.03 inches to about 0.038 inches and the inner diameter of the first portion 102a is about 0.04 inches. In some embodiments, the filter is placed in the first portion 102a of the center portion 102 such that the filter is affixed between the second portion 102b of the center portion 102 and the distal end 103.

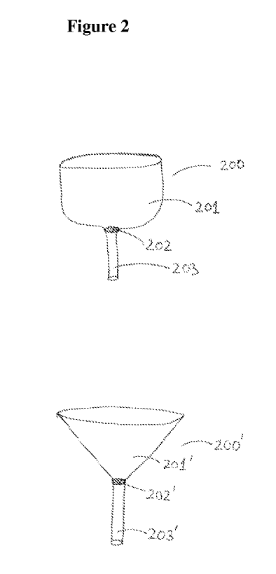

In another embodiment, the device is as shown in FIG. 2. The device 200 or 200′ comprises a proximal end 201 or 201′; a distal end 203 or 203′; and a filter 202 or 202′ affixed at the junction of the proximal end and the distal end. It is to be understood that design variations in the device are well within the skill of a person of ordinary skill in the art.

The filter comprises a plurality of pores wherein the pores have a diameter smaller than the diameter of the oocytes but larger than an individual cell of cumulus. This prevents oocytes to pass through the filter while permitting cumulus cells through it. In some embodiments, the plurality of pores have diameter from about 10 μm to about 90 μm; from about 10 μm to about 80 μm; from about 20 μm to about 70 μm; from about 20 μm to about 50 μm; from about 20 μm to about 30 μm; or about 30 μm.

The tube can be made of polymeric materials, including, but are not limited to, polycarbonate, polyester, terephthalate, or polyolefin. The filter can be made of polymeric materials, including, but not limited to, polycarbonate membrane, nylon, polyolefin etc. In some embodiments, the filter is made of polycarbonate membrane. Preferably, the tube and the filter are made of materials that are biocompatible and non-degradable in the presence of an aqueous solution containing a cumulus stripping agent.

In some embodiments, the tube comprises a series of screens or sieves to aid in stripping the cumulus from the oocyte. As the cumulus stripping agent softens the cumulus on the oocyte inside the tube, the cumulus may loosen and fluff up. The pulsitile flow of the aqueous solution comprising the cumulus stripping agent may drive the oocyte through the sieves from the one with largest mesh size to the smallest mesh size and then trap the oocytes on the filter. This may increase the efficiency of the stripping of the cumulus from the oocyte.

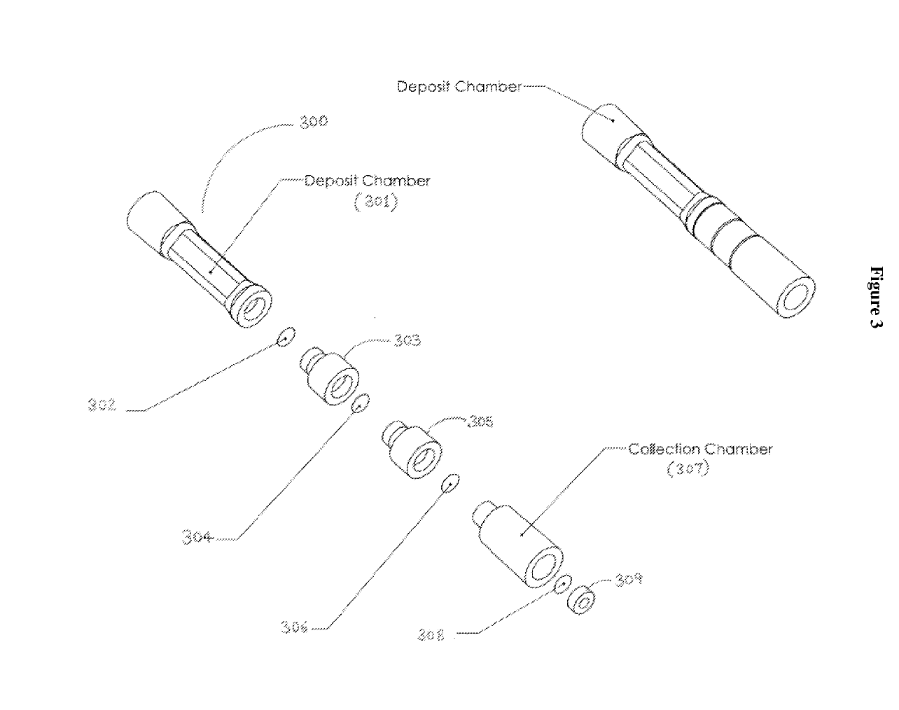

An example of the device, with multiple sieves and the filter, is as shown in FIG. 3. The tube 300 of the device comprises a deposit chamber 301 where the oocytes are deposited. In an embodiment, the oocytes are incubated in a solution containing a cumulus stripping agent inside the deposit chamber 301 for a time sufficient to allow degradation of the cumulus of the oocytes. The flow of the aqueous solution containing the cumulus stripping agent through the open end of the deposit chamber 301 drives the oocytes out of the deposit chamber 301 through the first sieve 302 and into the chamber 303. The flow of the solution further drives the oocytes out of the chamber 303 through the second sieve 304 into the chamber 305. Further flow of the solution drives the oocytes out of the chamber 305 through the third sieve 306 into the collection chamber 307. The filter 308 comprises a plurality of pores where the pores have a diameter smaller than the diameter of the oocytes but larger than an individual cell of cumulus. This prevents oocytes to pass through the filter 308 while permitting cumulus cells through it. Therefore, the oocytes are collected in the collection chamber 307 and the stripped cumulus is collected in the chamber 309. Alternatively, the stripped cumulus exits the device through the end 309. It is to be understood that the number of sieves shown in the device of FIG. 3 are for illustration purposes only. Depending on the number of oocytes, the flow of the aqueous solution, the length of the tube etc., the number of sieves may be increased or decreased. For example, the number of the sieves in the device may be anywhere from 1-10.

The mesh size of the sieves 302, 304, and 306 is large enough to pass the oocytes through the holes of the sieves. However, the pore size of the filter 308 prevents the passage of the oocytes through it.

In some embodiments, the mesh size of the sieve may differ depending on the nature of the oocytes. For example, the mesh size may be smaller for the mouse oocytes as compared to the human oocytes. In some embodiments, the first sieve size is 200 μm-300 μm; second sieve size is 150 μm-225 μm; third sieve size is 125 μm-185 μm; and filter is 40 μm. In some embodiments, the first sieve size is 200 μm-300 μm; second sieve size is 150 μm-200 μm; third sieve size is 125 μm-150 μm; and filter is 40 μm. In some embodiments, the first sieve size is 200 μm; second sieve size is 150 μm; third sieve size is 125 μm; and filter is 40 μm. In some embodiments, the first sieve size is 300 μm; second sieve size is 225 μm; third sieve size is 185 μm; and filter is 40 μm.

In some embodiments, the screen of the sieve is made of material including, but not limited to, polypropylene, polyester, polycarbonate, or stainless steel.

In some embodiments, the device is affixed to a source of an aqueous solution comprising a cumulus stripping agent. In some embodiments, the source is a syringe. In some embodiments, the syringe is capable of providing a pulsitile flow of the solution through the device. In some embodiments, the pulsitile flow through the syringe is powered by a stepper motor or a pump. The stepper motor or pump, typically, moves in discrete steps. For instance, 1 step of the stepper motor is ⅛ revolution. In some embodiments, the pulsitile flow through the syringe is powered by a server motor where an additional driver may be required for operating the motor. The server motor moves continuously in a non-discrete motion. It is to be understood that any means for generating a pulsitile flow of the aqueous solution through the tube may be used in the invention.

In some embodiments, the syringe pump provides a pulsitile flow of about 0.02-0.05 mL in a short burst then a 6 second rest, followed by another pulsitlile flow event that repeats 20-30 times.

### 3. Methods

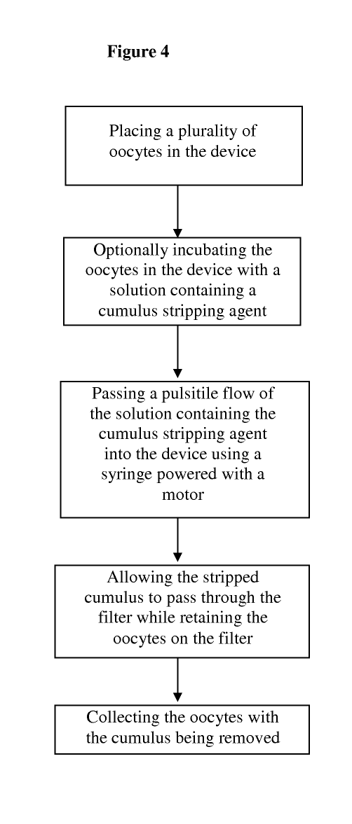

Disclosed herein are methods for stripping cumulus from a plurality of oocytes using the devices of the invention. The method can be generally described as shown in the flow chart in FIG. 4. The method may be initiated by placing the oocytes in the device. The oocytes may be placed in the proximal end of the device. Alternatively, the oocytes may be placed in the center portion of the device. The oocytes may also be placed on the filter affixed inside the tube. Optionally, the oocytes may be incubated in a solution containing a cumulus stripping agent for a time sufficient to allow degradation of a cumulus of the oocytes. This incubation may take place in the proximal end of the device or in the center portion of the device containing the oocytes. Alternatively, the incubation may be carried out outside the device and the oocytes may be placed in the tube after incubation for the cumulus stripping process.

A solution containing a cumulus stripping agent is then allowed to flow from the proximal end of the device to the distal end of the device. This solution may be passed using any means that can pass the solution in the device, such as, but not limited to, syringe, dropper etc. Preferably, the solution is allowed to flow through the device in pulses. In some embodiments, a syringe capable of providing a pulsitile flow of the solution through the device is used. The pulsitile flow of the solution may be achieved using a stepper motor or a server motor attached to the syringe. The motor may be pre-programmed to provide a definite pulsitile flow of the solution through the device. In some embodiments, the pulsitile flow of the solution is every 5 to 10 seconds with a rest in between. This pulsitile flow may be repeated less than about 100 times; less than about 80 times; less than about 50 times or less than about 30 times. In some embodiments, the pulsitile flow may be repeated from about 10 to 30 times. In some embodiments, the syringe pump provides a pulsitile flow of approximately 0.02 mL in a short burst then a 6 second rest, followed by another pulsitlile flow event repeated 20 times. It will be understood that the number of times the pulsitile flow is repeated may depend on the number of oocytes, concentration of the cumulus stripping agent or length of the device etc.

The stripped cumulus may pass through the filter and out of the device through the distal end whereas the oocytes remain on the filter. The oocytes may be collected by removing the filter from the device. Alternatively, back washing the filter will release the oocytes without manual intervention.

The solution containing the cumulus stripping agent aided by the pulsitile flow, strips the cumulus off of the oocyte. Examples of cumulus stripping agent include, but are not limited to, bovine-derived hyaluronidase (Hyase), recombinant human derived enzyme product (Cumulase) (see Nagy, Z. P. et al. supra); and plant enzyme (coronase). In some embodiments, solution containing the cumulus stripping agent further includes, but is not limited to, buffered saline or any media simulating physiologic osmolality and replicating the environment in the fallopian tubes such as Irvine scientific HTF (human tubal fluid) or M-HTF (modified-HTF) solution.

In some embodiments, after the removal of the cumulus from the oocytes, the oocytes are optionally washed with a rinse solution. In a preferred embodiment, the oocytes after cumulus removal are washed with rinse solution once or multiple times. The rinse solution contains, but is not limited to, HSA (human serum albumin) in HTF or m-HTF.

Accordingly, in another aspect of the invention, there is provided a method for stripping cumulus from a plurality of oocytes which method comprises:

1) placing the plurality of oocytes on a filter affixed inside a tube, wherein the filter comprises a plurality of pores wherein the pores have a diameter smaller than a diameter of the oocytes but larger than an individual cells of cumulus thereby restricting passage of said oocytes through the filter while permitting passage of the cumulus cells there through; and 2) flowing an effective amount of solution comprising a cumulus stripping agent over the oocytes for a time sufficient to strip the cumulus from the oocytes.

In one embodiment of the methods of the invention, the tube comprises an open proximal end, an open distal end and a center portion with a lumen running from the proximal to the distal end of a defined diameter which lumen permits flow of a fluid through said device wherein the diameter of the center portion is narrower as compared to the diameter of the distal end thereby providing a constriction. In some embodiments, the filter is affixed inside the tube at the point of constriction.

In an alternative embodiment, the diameter of the center portion is wider than the diameter of the distal or the proximal end of the device and the filter is affixed inside the tube in the wider center portion.

In one embodiment, the solution flows from the proximal end to the distal end of the tube.

In one embodiment, the method further comprises:

3) allowing a stripped cumulus to pass through the filter while retaining the oocytes in on the filter of the device; and

4) optionally repeating step 2) and step 3), thereby stripping the cumulus from the oocytes.

In one embodiment, the step 3) is followed by a rest. In one embodiment, the rest is for about 5 to about 10 seconds.

In one embodiment of the methods of the invention, the flow of the solution is provided by a syringe containing the solution. In one embodiment, the flow of the solution is a pulsitile flow of the solution.

### 4. Kit of Parts

In one aspect of the invention, there is provided kit of parts comprising the device of the invention and the syringe that flows solution into the device. The kit further comprises a motor that may be attached to the syringe or may be provided separately to be attached to the syringe at the time of operation. The kit may further comprise a container, such as a bottle, an ampule or a syringe, containing a solution of cumulus stripping agent. Alternatively, the cumulus stripping agent and the aqueous solution may be provided in separate containers to be mixed at the time of use. The kit may further comprise an instruction sheet for using the parts. The kit may also comprise usual operational tools, such as forceps, gloves, petri dish, etc.

### EXAMPLE

The following example is provided to illustrate certain aspects of the present invention and to aid those of skill in the art in practicing the invention. The example is in no way to be considered to limit the scope of the invention. Any methods that are functionally equivalent are within the scope of the invention. Various modifications of the invention in addition to those described herein will become apparent to those skilled in the art from the foregoing description and accompanying figures. Such modifications fall within the scope of the appended claims.

In these examples and elsewhere, abbreviations have the following meanings:

| | |
|---|---|
| g/L | gram/liter |
| HSA | human serum albumin |
| IVF | in vitro fertilization |
| m-HTF | modified human tubal fluid |
| mg | milligram |
| μm | micrometer |
| mL | milliliter |
| mM | millimolar |
| IU/mL | International units/milliliter |

### Example 1

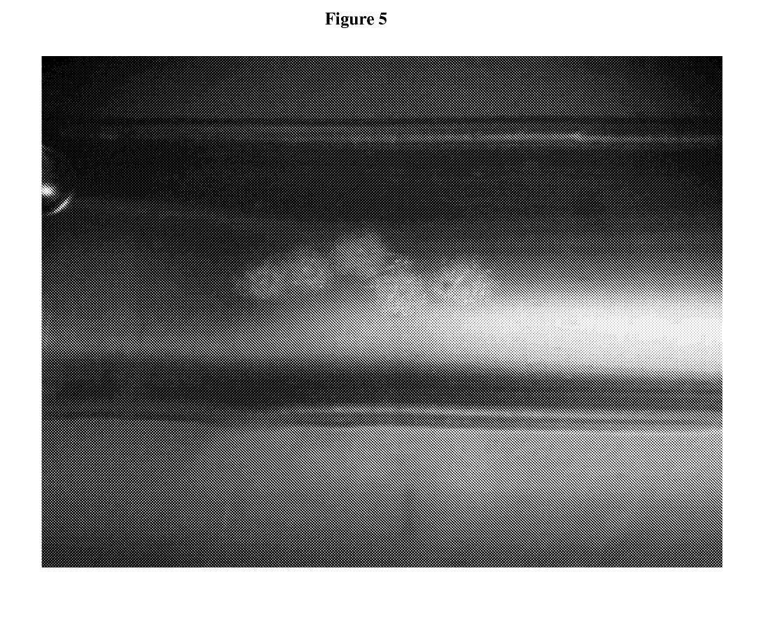

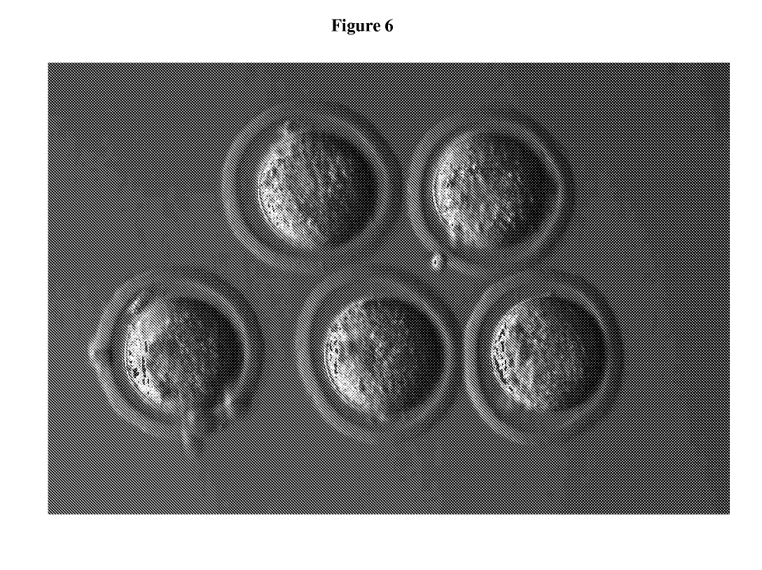

A group of five mouse oocytes with cumulus attached were received from Embryotech Laboratories (Haverhill, Mass.). The oocytes were loaded into the device containing a 80 IU/mL solution of hyaluronidase enzyme (Irvine Scientific P/N 90101) or a solution prepared by dissolving 10 mg of hyaluronidase enzyme in 42.12 mL m-HTF (Irvine Scientific P/N 90126). The device was attached to a 1 mL syringe holding the enzyme solution. After 2 minutes at room temperature, the syringe pump was activated. A total of 0.4 mL was used in 15 bolus infusions of 0.02 mL per bolus. After the treatment with the enzyme solution, the oocytes are rinsed with a rinse solution containing 4% human serum albumin (Irvine Scientific, #9988) in m-HTF. FIG. 5 shows the five oocytes with cumulus attached to the oocytes. FIG. 6 shows the five oocytes after stripping of the cumulus.

### Example 2

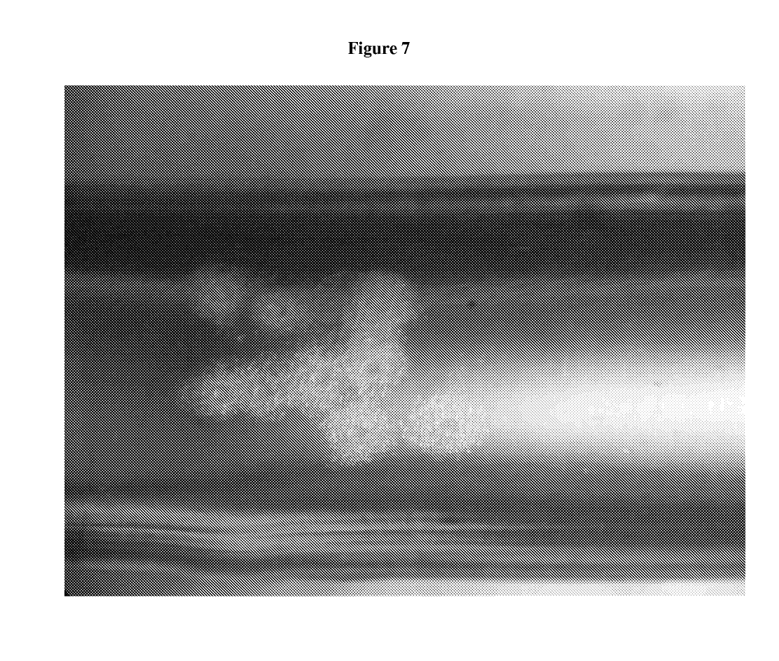

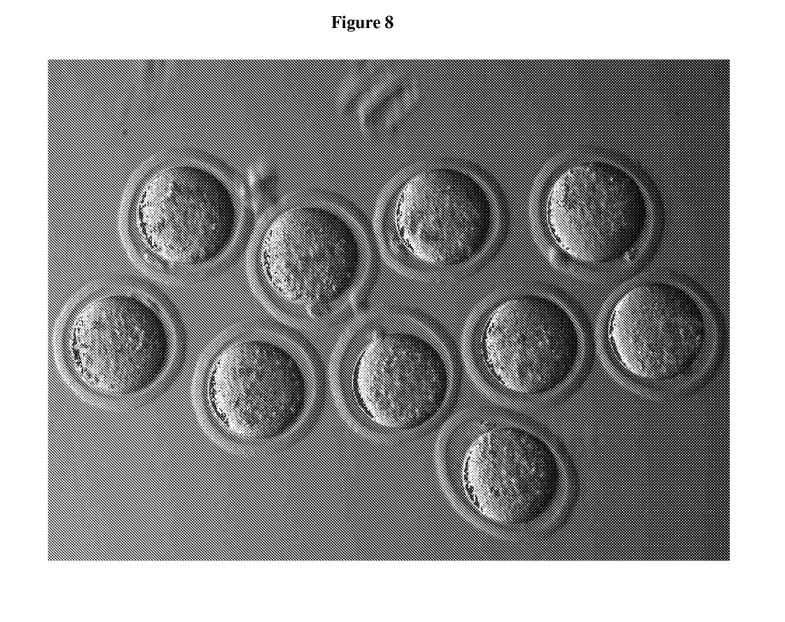

A group of ten mouse oocytes with cumulus attached were received from Embryotech Laboratories (Haverhill, Mass.). The oocytes were loaded into the device containing a 80 IU/mL solution of hyaluronidase enzyme or a solution prepared by dissolving 10 mg of hyaluronidase enzyme in 42.12 mL m-HTF. The device was attached to a 1 mL syringe holding the enzyme solution. After 2 minutes at room temperature, the syringe pump was activated. A total of 0.4 mL was used in 15 bolus infusions of 0.02 ml per bolus. After the treatment with the enzyme solution, the oocytes are rinsed with a rinse solution containing 4% human serum albumin (Irvine Scientific, #9988) in m-HTF. FIG. 7 shows the ten oocytes with cumulus attached to the oocytes. FIG. 8 shows the ten oocytes after stripping of the cumulus.

### Example 3

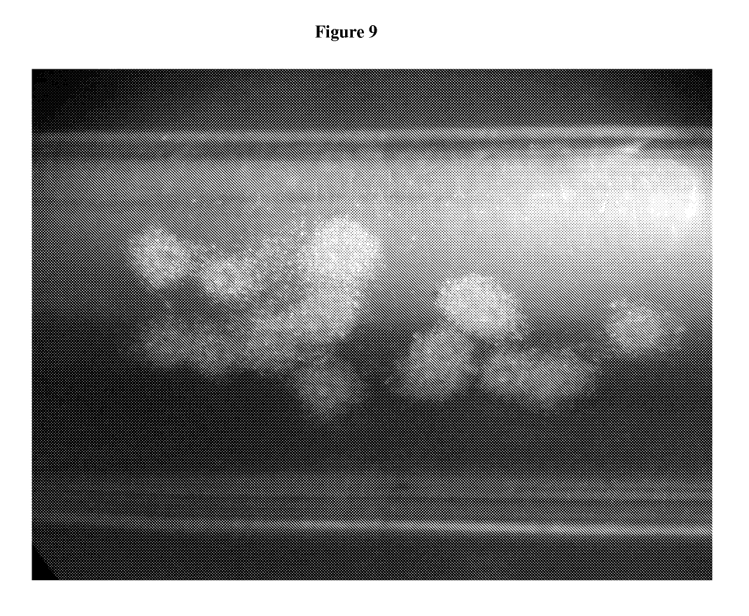

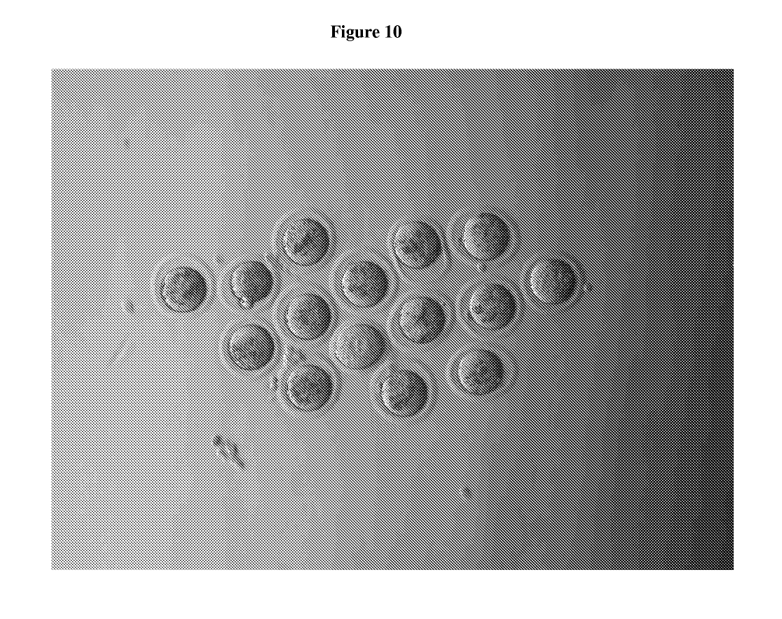

A group of fifteen mouse oocytes with cumulus attached were received from Embryotech Laboratories (Haverhill, Mass.). The oocytes were loaded into the device containing a 80 IU/mL solution of hyaluronidase enzyme or a solution prepared by dissolving 10 mg of hyaluronidase enzyme in 42.12 mL m-HTF. The device was attached to a 1 mL syringe holding the enzyme solution. After 2 minutes at room temperature, the syringe pump was activated. A total of 0.4 mL was used in 15 bolus infusions of 0.02 mL per bolus. After the treatment with the enzyme solution, the oocytes are rinsed with a rinse solution containing 4% human serum albumin (Irvine Scientific, #9988) in m-HTF. FIG. 9 shows the fifteen oocytes with cumulus attached to the oocytes. FIG. 10 shows the fifteen oocytes after stripping of the cumulus. [0080] It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A device adapted for stripping cumulus from a plurality of oocytes, which device comprises:
a tube; and
a filter affixed inside the tube, which filter comprises a plurality of pores having a diameter smaller than a diameter of said oocytes but larger than an individual cells of cumulus thereby restricting passage of said oocytes through the filter while permitting passage of the cumulus cells there through.

2. The device of claim 1, wherein the tube has a constriction and the filter is affixed inside the tube at the point of constriction.

3. The device of claim 1, wherein the tube has a wider center portion and the filter is affixed inside the tube in the wider center portion.

4. The device of claim 1, wherein said tube comprises an open proximal end, an open distal end and a center portion with a lumen running from the proximal to the distal end of a defined diameter which lumen permits flow of a fluid through said device wherein the diameter of the center portion is narrower as compared to the diameter of the distal end providing a constriction.

5. The device of claim 1, wherein the filter holds a plurality of oocytes.

6. The device of claim 1, wherein the filter is replaceable.

7. (canceled)

8. (canceled)

9. The device of claim 1, wherein the plurality of pores have diameters from about 10 μm to about 90 μm.

10. (canceled)

11. (canceled)

12. The device of claim 1, wherein the tube is about 0.5 inch to about 10 inches long.

13. (canceled)

14. (canceled)

15. The device of claim 4, wherein the center portion of the tube has an inner diameter from about 0.01 inch to about 0.1 inch.

16. (canceled)

17. The device of claim 1, wherein the plurality of oocytes are mammalian oocytes.

18. (canceled)

19. The device according to claim 1 to which device is affixed to a source of an aqueous solution comprising a cumulus stripping agent.

20. (canceled)

21. (canceled)

22. (canceled)

23. A method for stripping cumulus from a plurality of oocytes which method comprises:
1) placing the plurality of oocytes on a filter affixed inside a tube, wherein the filter comprises a plurality of pores wherein the pores have a diameter smaller than a diameter of the oocytes but larger than an individual cells of cumulus thereby restricting passage of said oocytes through the filter while permitting passage of the cumulus cells there through; and
2) flowing an effective amount of solution comprising a cumulus stripping agent over the oocytes for a time sufficient to strip the cumulus from the oocytes.

24. The method of claim 23, wherein the tube comprises an open proximal end, an open distal end and a center portion with a lumen running from the proximal to the distal end of a defined diameter which lumen permits flow of a fluid through said device wherein the diameter of the center portion is narrower as compared to the diameter of the distal end thereby providing a constriction.

25. The method of claim 24, wherein the filter is affixed inside the tube at the point of constriction.

26. The method of claim 23, wherein the solution flows from the proximal end to the distal end of the tube.

27. The method of claim 23, wherein the method further comprises:
3) allowing a stripped cumulus to pass through the filter while retaining the oocytes in on the filter of the device; and
4) optionally repeating step 2) and step 3), thereby stripping the cumulus from the oocytes.

28. The method of claim 27, wherein the step 3) is followed by a rest.

29. (canceled)

30. (canceled)

31. The method of claim 23, wherein the flow of the solution is a pulsitile flow of the solution.

32. The method of claim 23, wherein the solution comprises a cumulus stripping agent.

33. The method of claim 23, further comprising incubating the oocytes in a solution comprising a cumulus stripping agent for a time sufficient to allow degradation of a cumulus of the oocytes before step 1).
